(19) United States
(12) Patent Application Publication
(10) Pub. No.: US 2024/0252149 A1
(43) Pub. Date: Aug. 1, 2024

(54) ULTRASOUND INFORMATION PROCESSING APPARATUS, ULTRASOUND DIAGNOSTIC SYSTEM, AND ULTRASOUND DIAGNOSTIC APPARATUS

(71) Applicant: FUJIFILM Healthcare Corporation, Kashiwa-shi (JP)

(72) Inventors: Yu Zhao, Chiba (JP); Eiji Kasahara, Chiba (JP); Akira Kusakabe, Chiba (JP); Suguru Ishiguro, Chiba (JP)

(21) Appl. No.: 18/416,018

(22) Filed: Jan. 18, 2024

(30) Foreign Application Priority Data
Jan. 27, 2023 (JP) 2023-010676

(51) Int. Cl. A61B 8/08; A61B 8/00

(52) U.S. Cl. CPC A61B 8/5223; A61B 8/463; A61B 8/465; A61B 8/565

(57) ABSTRACT

An information processing apparatus includes an information processing unit that acquires ultrasound diagnostic data generated by an ultrasound diagnostic apparatus. The information processing unit acquires an activation command from the ultrasound diagnostic apparatus, and transmits operation command list for an execution application, which is activated by the activation command, among a plurality of applications for the ultrasound diagnostic data to the ultrasound diagnostic apparatus. The operation command list includes an operation command for operating the execution application and information for constructing a man-machine interface for operating the execution application. The ultrasound diagnostic apparatus constructs the man-machine interface corresponding to operation command information.

### CROSS-REFERENCE TO RELATED APPLICATION

The present application claims priority from Japanese patent application 2023-010676 filed on Jan. 27, 2023, the content of which is hereby incorporated by reference into this application.

### BACKGROUND OF THE INVENTION

### 1. Field of the Invention

The present disclosure relates to an ultrasound information processing apparatus, an ultrasound diagnostic system, and an ultrasound diagnostic apparatus, and more particularly to a man-machine interface.

### 2. Description of the Related Art

There is a technique in which an information processing apparatus is connected to an outside of an ultrasound diagnostic apparatus by wired communication or wireless communication, the information processing apparatus is caused to execute additional processing, and information generated by the information processing apparatus is displayed by the ultrasound diagnostic apparatus. Examples of the processing executed by the information processing apparatus include processing of superimposing an MRI image or the like on a moving image of a real-time ultrasound image, and VFM or AI analysis using moving image data.

JP2013-111100A, JP2020-142017A, and JP2015-198777A describe techniques related to the present disclosure. JP2013-111100A describes an ultrasound diagnostic apparatus that transmits medical information to an external terminal. The ultrasound diagnostic apparatus sets a transmission condition based on at least one of information about a communication environment of the ultrasound diagnostic apparatus or information about an internal state of the ultrasound diagnostic apparatus, and transmits subset data generated based on the transmission condition to the external terminal. The subset data includes a medical image and measurement information obtained from the medical image, as medical information.

JP2020-142017A describes an ultrasound diagnostic apparatus in which preparation software is stored in an ultrasound probe. The processing according to the preparation software is executed by a terminal device provided in the ultrasound diagnostic apparatus. The preparation software is a program for installing appropriate software related to an ultrasound diagnosis on the terminal device. JP2015-198777A describes a technique of measuring a blood flow velocity by a Doppler method.

### SUMMARY OF THE INVENTION

A plurality of applications are installed in the information processing apparatus provided outside the ultrasound diagnostic apparatus. A man-machine interface corresponding to each application is installed in the ultrasound diagnostic apparatus. In a case in which the application installed in the information processing apparatus is added, it is necessary to install a new man-machine interface in the ultrasound diagnostic apparatus. Therefore, in a case in which the application installed in the information processing apparatus is added, the work of adding software to the ultrasound diagnostic apparatus may be troublesome.

An object of the present disclosure is to facilitate work of adding an application installed in an information processing apparatus in a case in which the information processing apparatus is provided outside an ultrasound diagnostic apparatus.

An aspect of the present disclosure relates to an ultrasound information processing apparatus comprising: an information processing unit that acquires ultrasound diagnostic data generated by an ultrasound diagnostic apparatus by transmission and reception of ultrasound, in which the information processing unit acquires an activation command from the ultrasound diagnostic apparatus, and transmits operation command information for an execution application, which is activated by the activation command, among a plurality of applications for the ultrasound diagnostic data to the ultrasound diagnostic apparatus, the operation command information includes an operation command for operating the execution application and information for constructing a man-machine interface for operating the execution application, and the ultrasound diagnostic apparatus constructs the man-machine interface corresponding to the operation command information.

In one embodiment, the man-machine interface provides an operation portion used with execution of the execution application to a user.

In one embodiment, the operation command information includes information for constructing a graphical user interface used with execution of the execution application, and the ultrasound diagnostic apparatus constructs the graphical user interface as the man-machine interface.

In one embodiment, the information processing unit displays an operation procedure for the operation command on display equipment.

In one embodiment, the information processing unit displays an image including a part or all of images displayed by the ultrasound diagnostic apparatus on display equipment.

Another aspect of the present disclosure relates to an ultrasound diagnostic system comprising: the ultrasound information processing apparatus; and the ultrasound diagnostic apparatus.

Still another aspect of the present disclosure relates to an ultrasound diagnostic apparatus comprising: a controller that transmits ultrasound diagnostic data generated by transmission and reception of ultrasound to an information processing apparatus, and acquires external processing data in which the ultrasound diagnostic data is processed from the information processing apparatus, in which the controller acquires operation command information for an application for the ultrasound diagnostic data, which is installed in the information processing apparatus, from the information processing apparatus, and constructs a man-machine interface corresponding to the operation command information.

In one embodiment, the man-machine interface provides an operation portion used with execution of the application to a user.

In one embodiment, the operation command information includes information for constructing a graphical user interface used with execution of the application, and the controller constructs the graphical user interface as the man-machine interface.

In one embodiment, the controller displays an image including a part or all of images displayed by the ultrasound diagnostic apparatus on the information processing apparatus.

According to the aspects of the present disclosure, it is possible to facilitate the work of adding the application installed in the information processing apparatus in a case in which the information processing apparatus is provided outside the ultrasound diagnostic apparatus.

### BRIEF DESCRIPTION OF THE DRAWINGS

FIG. 1 is a diagram showing an ultrasound diagnostic system according to an embodiment of the present disclosure.

FIG. 2 is a diagram showing the ultrasound diagnostic system according to the embodiment of the present disclosure.

FIG. 3 is a diagram showing configurations of an ultrasound diagnostic apparatus and an information processing apparatus.

FIG. 4 is a sequence chart in which the information processing apparatus executes a j-th application as an execution application.

FIG. 5 is a diagram showing an example of a graphical user interface image displayed on a display device.

FIG. 6 is a diagram showing an example of the graphical user interface image displayed on the display device.

FIG. 7 is a diagram showing two examples of the graphical user interface image displayed on the display device.

FIG. 8 is a diagram showing an example of a GUI image displayed on each of a display, a touch panel, and a tablet computer.

FIG. 9 is a diagram showing an example of the GUI image displayed on each of the display, the touch panel, and the tablet computer.

### DESCRIPTION OF THE PREFERRED EMBODIMENTS

An embodiment of the present disclosure will be described with reference to the accompanying drawings. The same components shown in a plurality of drawings are denoted by the same reference numerals, and the description thereof will be omitted.

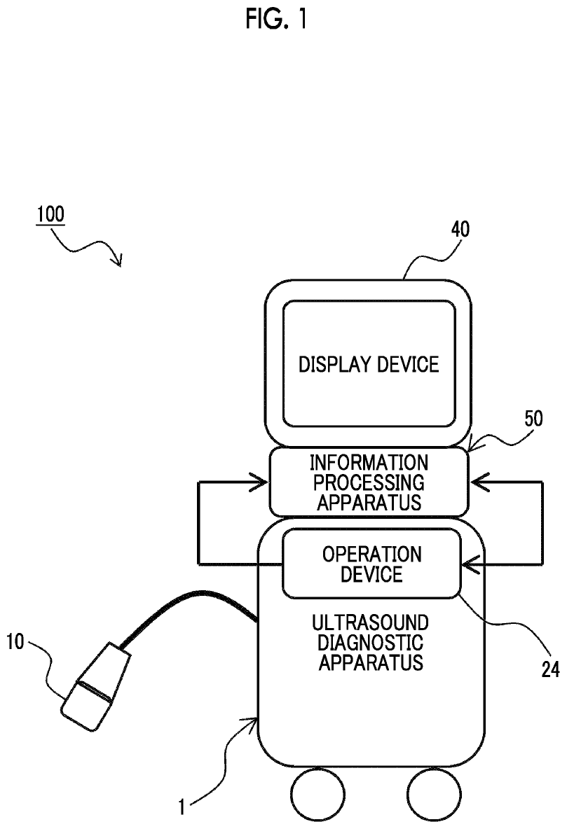

FIG. 1 shows an ultrasound diagnostic system 100 according to an embodiment of the present disclosure. The ultrasound diagnostic system 100 comprises an ultrasound diagnostic apparatus 1 and an information processing apparatus 50 (ultrasound information processing apparatus). The ultrasound diagnostic apparatus 1 and the information processing apparatus 50 may be connected to each other by wire or wirelessly. In a case in which a subject is diagnosed, an ultrasound probe 10 provided in the ultrasound diagnostic apparatus 1 is in a state in which a transmission/reception surface at a distal end of the ultrasound probe 10 is in contact with a surface of the subject. The ultrasound diagnostic apparatus 1 causes the ultrasound probe 10 to transmit ultrasound. The ultrasound transmitted from the ultrasound probe 10 is reflected in the subject and is received by the ultrasound probe 10. The ultrasound probe 10 converts the received ultrasound into a reception signal, which is an electrical signal.

The ultrasound diagnostic apparatus 1 generates image data based on the reception signal. The ultrasound diagnostic apparatus 1 causes the information processing apparatus 50 to execute additional image processing, and displays the image data generated by the information processing apparatus 50 on a display device 40 of the ultrasound diagnostic apparatus 1. The ultrasound diagnostic apparatus 1 may display an image based on the image data, which is not processed by the information processing apparatus 50, on the display device 40.

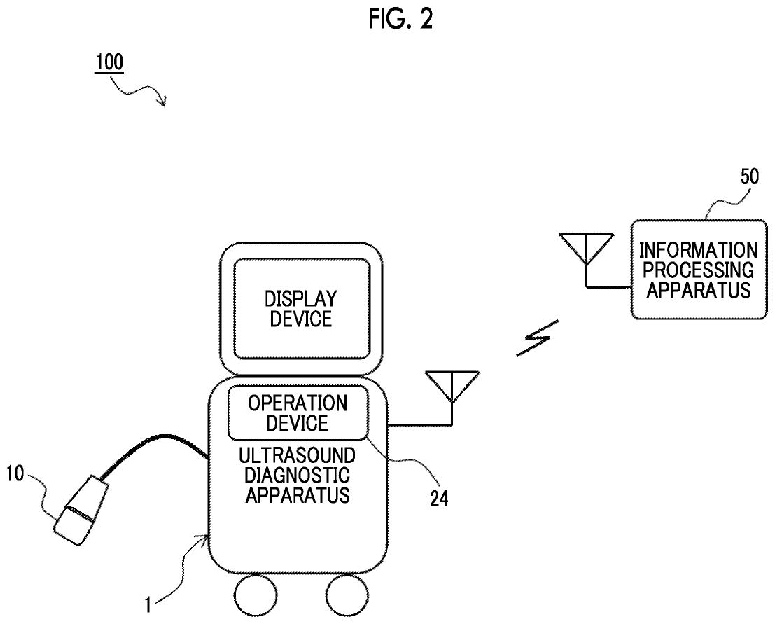

FIG. 2 shows an embodiment in which the information processing apparatus 50 and the ultrasound diagnostic apparatus 1 are connected to each other wirelessly and the wireless communication is performed between the information processing apparatus 50 and the ultrasound diagnostic apparatus 1. The information processing apparatus 50 may be a personal computer, a workstation, a tablet computer, a smartphone, and the like having a wireless communication function.

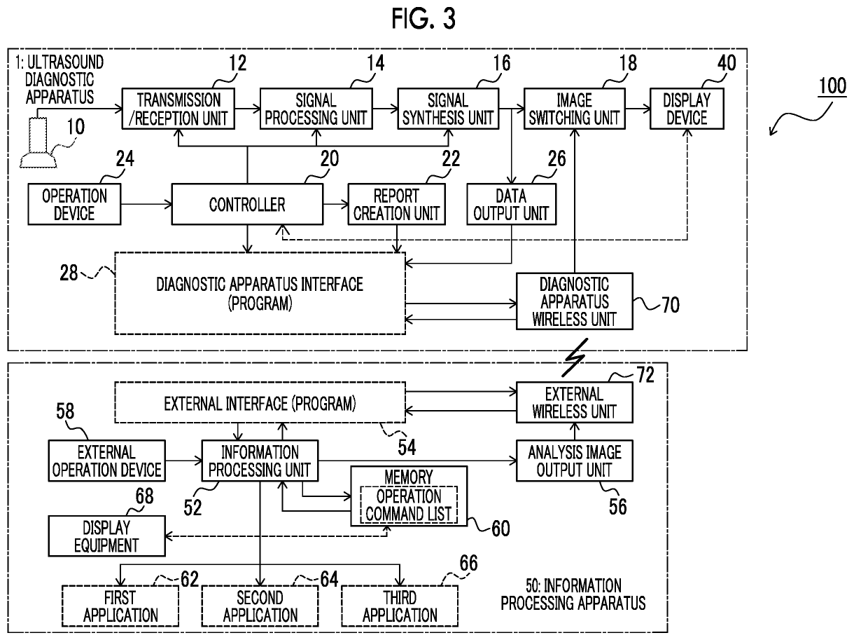

FIG. 3 shows a configuration of the ultrasound diagnostic system 100. The ultrasound diagnostic system 100 comprises the ultrasound diagnostic apparatus 1 and the information processing apparatus 50. The ultrasound diagnostic apparatus 1 comprises an ultrasound probe 10, a transmission/reception unit 12, a signal processing unit 14, a signal synthesis unit 16, an image switching unit 18, a display device 40, a controller 20, a report creation unit 22, an operation device 24, a data output unit 26, a diagnostic apparatus interface 28, and a diagnostic apparatus wireless unit 70.

The signal processing unit 14, the signal synthesis unit 16, the image switching unit 18, the controller 20, the report creation unit 22, and the data output unit 26 may include a processor and an electronic circuit that execute a program to realize functions of these components (signal processing unit 14, signal synthesis unit 16, image switching unit 18, controller 20, report creation unit 22, and data output unit 26). The image switching unit 18 may include an electronic circuit that realizes a communication function.

The controller 20 executes entire control of the ultrasound diagnostic apparatus 1. The operation device 24 may comprise a button, a lever, a keyboard, a mouse, and the like. The operation device 24 may be a touch panel provided on the display device 40. The controller 20 may execute the control of the ultrasound diagnostic apparatus 1 based on an operation by a user. The controller 20 transmits and receives information for the information processing apparatus 50 to execute an application to and from the information processing apparatus 50 via the diagnostic apparatus interface 28 and the diagnostic apparatus wireless unit 70.

Here, the diagnostic apparatus interface 28 is constructed by the controller 20 executing a diagnostic apparatus interface program. In FIG. 3, for convenience of description, the diagnostic apparatus interface 28 is drawn by a broken line separately from the controller 20. The diagnostic apparatus interface 28 is operated together with the diagnostic apparatus wireless unit 70. The diagnostic apparatus interface 28 may be operated together with an electronic circuit that performs the wired communication with the information processing apparatus 50.

The ultrasound probe 10 comprises a plurality of ultrasound oscillators. The transmission/reception unit 12 outputs a transmission signal, which is an electrical signal, to the plurality of ultrasound oscillators. Each ultrasound oscillator converts the transmission signal into the ultrasound and transmits the transmission signal to the subject. The transmission/reception unit 12 adjusts a delay time of the transmission signal output to each ultrasound oscillator, to form an ultrasound beam in a specific direction.

Each ultrasound oscillator receives the ultrasound reflected in the subject, converts the ultrasound into the reception signal which is the electrical signal, and outputs the reception signal to the signal processing unit 14. The signal processing unit 14 adjusts the delay time of the reception signal output from each ultrasound oscillator so that the reception signals of the ultrasound coming from a direction in which the ultrasound beam is directed strengthen each other, and adds and totals the reception signals after the delay time adjustment. The signal processing unit 14 outputs a phasing addition signal generated in this manner to the signal synthesis unit 16.

It should be noted that the transmission/reception unit 12 changes the delay time of the transmission signal output to each ultrasound oscillator so that an inside of a specific observation cross section of the subject is scanned with the ultrasound beam. In addition, the signal processing unit 14 changes the delay time of the reception signal output from each ultrasound oscillator so that the phasing addition signal corresponding to the direction in which the inside of the subject is scanned with the ultrasound beam is generated, and adds and totals the reception signals after the delay time adjustment.

The signal synthesis unit 16 generates B-mode image data based on the phasing addition signal acquired for each direction in the observation cross section, and outputs the B-mode image data to the image switching unit 18. The transmission/reception unit 12, the signal processing unit 14, and the signal synthesis unit 16 sequentially generate the B-mode image data at a predetermined frame rate with an elapse of time. Here, the frame rate refers to the number of B-mode images generated per unit time.

In basic display processing executed by the ultrasound diagnostic system 100, the image switching unit 18 outputs the B-mode image data sequentially generated with the elapse of time to the display device 40. The display device 40 displays an image based on the B-mode image data sequentially generated with the elapse of time, that is, a real-time image of the B-mode image.

In the above description, the processing of scanning the inside of the subject with the ultrasound beam and generating the B-mode image based on the phasing addition signal corresponding to the ultrasound beam in each direction is shown. The ultrasound diagnostic apparatus 1 may execute an operation of a Doppler mode in which a blood flow velocity is obtained based on a difference of the frequency of the phasing addition signal (Doppler shift) from a frequency of the transmission signal. The controller 20 generates, for example, Doppler data indicating the blood flow velocity in a predetermined range defined on the ultrasound beam in each direction.

The controller 20 outputs ultrasound diagnostic data such as the B-mode image data and the Doppler data acquired for the ultrasound beam in each direction to the report creation unit 22. The report creation unit 22 generates an ultrasound examination report based on the ultrasound diagnostic data. The ultrasound examination report is data indicating information such as a size and a shape of tissue, and whether or not findings are recognized. The ultrasound examination report may be generated in a representation format corresponding to the tissue that is a diagnosis target.

The ultrasound diagnostic system 100 may execute the following practical application display processing. The signal synthesis unit 16 outputs the ultrasound diagnostic data sequentially generated with the elapse of time to the data output unit 26. The data output unit 26 outputs the ultrasound diagnostic data sequentially generated with the elapse of time to the diagnostic apparatus interface 28. The diagnostic apparatus interface 28 transmits the ultrasound diagnostic data to the information processing apparatus 50 by the wireless communication via the diagnostic apparatus wireless unit 70.

An application for performing specific processing on the ultrasound diagnostic data is installed in the information processing apparatus 50. This application includes a first application 62 that generates streaming image data based on the B-mode image data that is sequentially generated with the elapse of time. The streaming image data refers to data for displaying the real-time image based on the B-mode image data sequentially generated with the elapse of time.

In a case in which the information processing apparatus 50 executes the application, the information processing apparatus 50 generates application analysis data (hereinafter, referred to as APP analysis data) as external processing data. “APP” is an abbreviation for application. The APP analysis data may be data indicating a numerical value or image data. The information processing apparatus 50 transmits the APP analysis data to the ultrasound diagnostic apparatus 1, as described later.

A configuration of the information processing apparatus 50 and processing executed by the information processing apparatus 50 will be described in detail. The information processing apparatus 50 comprises an information processing unit 52, an external interface 54, an analysis image output unit 56, an external operation device 58, a memory 60, the first application 62, a second application 64, a third application 66, display equipment, and an external wireless unit 72. The information processing apparatus 50 may include a processor and an electronic circuit that execute a program to realize a function of each of the components (information processing unit 52, external interface 54, analysis image output unit 56, first application 62, second application 64, third application 66). The analysis image output unit 56 performs the wireless communication with the ultrasound diagnostic apparatus 1 via the external wireless unit 72. The analysis image output unit 56 may be operated together with an electronic circuit that performs the wired communication with the ultrasound diagnostic apparatus 1, or may perform the wired communication with the ultrasound diagnostic apparatus 1.

The information processing apparatus 50 may be a computer such as a personal computer, a workstation, a tablet computer, or a smartphone. The information processing unit 52 performs entire control of the information processing apparatus 50. In a case in which the information processing apparatus 50 is a personal computer, a workstation, and the like, the external operation device 58 may be a keyboard, a mouse, and the like. The information processing apparatus 50 comprises display equipment 68 such as a display. In a case in which the display equipment 68 is, for example, a tablet computer or a smartphone, the external operation device 58 may be a touch panel configured on the display equipment 68. It should be noted that the display equipment 68 does not always have to be provided.

The information processing unit 52 may control the information processing apparatus 50 according to an operation of the external operation device 58 by the user. The external interface 54 transmits and receives signals to and from the diagnostic apparatus interface 28 by the wireless communication via the external wireless unit 72 and the diagnostic apparatus wireless unit 70. In FIG. 3, for convenience of description, the external interface 54 is drawn by a broken line separately from the information processing unit 52.

Here, the external interface 54 is a component constructed by the information processing unit 52 executing an external interface program. The external interface 54 is operated together with the external wireless unit 72. The external interface 54 may be operated together with an electronic circuit that performs the wired communication with the ultrasound diagnostic apparatus 1, or may perform the wired communication with the ultrasound diagnostic apparatus 1.

Each of the first application 62 to the third application 66 is a component constructed by the information processing unit 52 executing a first application program to a third application program. In FIG. 3, for convenience of description, the first application 62 to the third application 66 are drawn by a broken line separately from the information processing unit 52.

The first application 62 to the third application 66 have different information that is transmitted to and received from the ultrasound diagnostic apparatus 1. On the other hand, the external interface 54 and the diagnostic apparatus interface 28 are interfaces common to the first application 62 to the third application 66, and transmit and receive the information corresponding to the operation of each application. That is, each of the first application 62 to the third application 66 does not individually have an interface for transmitting and receiving the information to and from the ultrasound diagnostic apparatus 1, and transmits and receives the information to and from the ultrasound diagnostic apparatus 1 via the external interface 54 and the diagnostic apparatus interface 28 constructed in common to the first application 62 to the third application 66.

The information processing unit 52 reads an application list from the memory 60 before the ultrasound diagnostic apparatus 1 executes the processing of generating the ultrasound diagnostic data. The application list may be information in which an activation command is associated with information for identifying each application installed in the information processing apparatus 50. The activation command is a command for activating the application by the operation of the user in the ultrasound diagnostic apparatus 1. The application list is stored in advance in the memory 60 according to the application installed in the information processing apparatus 50, and is updated in a case in which the application is newly installed in the information processing apparatus 50.

The information processing unit 52 transmits the application list to the controller 20 via the external interface 54, the external wireless unit 72, the diagnostic apparatus wireless unit 70, and the diagnostic apparatus interface 28.

The controller 20 displays the application list on the display device 40. In a case in which an operation of selecting any one of the first application 62 to the third application 66 is performed by the user on the operation device 24 while the application list is displayed on the display device 40 or after the display ends, the controller 20 transmits the activation command corresponding to the selected application. That is, the controller 20 transmits the activation command to the information processing unit 52 via the diagnostic apparatus interface 28, the diagnostic apparatus wireless unit 70, the external wireless unit 72, and the external interface 54. The information processing unit 52 activates an application corresponding to the activation command as the execution application.

After the execution application is activated, the information processing unit 52 reads an operation command list from the memory 60 as operation command information including the information listing operation commands used in the execution application. The operation command is information transmitted from the ultrasound diagnostic apparatus 1 to the information processing apparatus 50 according to the operation by the user in order to operate the execution application. The operation command list is stored in the memory 60 in a case in which the application is installed in the information processing apparatus 50. In the present embodiment, the operation command list is stored in the memory 60 for each of the first application 62, the second application 64, and the third application 66.

The operation command list includes MMI construction information for the ultrasound diagnostic apparatus 1 to construct a man-machine interface (hereinafter, may be referred to as MMI) for operating the operation command, in addition to the information listing the operation commands. Here, MMI is an abbreviation for man machine interface. The MMI provides an operation portion that is used with the execution of the execution application to the user. The operation portion may be, for example, a button displayed on the display device 40 and operated by a mouse or a user's finger. The operation command list may include the MMI construction information for the ultrasound diagnostic apparatus 1 to construct a graphical user interface (hereinafter, may be referred to as GUI) as the MMI. Here, GUI is an abbreviation for graphical user interface.

The information processing unit 52 transmits the operation command list to the controller 20 via the external interface 54, the external wireless unit 72, the diagnostic apparatus wireless unit 70, and the diagnostic apparatus interface 28. The controller 20 displays a graphical user interface image (GUI image) on the display device 40, as an image for operating the operation command.

In a case in which an operation for the operation command is performed by the user on the operation device 24 while the GUI image is displayed on the display device 40, the controller 20 transmits the operation command corresponding to the operation. That is, the controller 20 transmits the operation command to the information processing unit 52 via the diagnostic apparatus interface 28, the diagnostic apparatus wireless unit 70, the external wireless unit 72, and the external interface 54. The information processing unit 52 executes processing of the execution application corresponding to the operation command.

The application installed in the information processing apparatus 50 can be added or removed. In a case in which the application is added or removed, the application list stored in the memory 60 is updated. In addition, the operation command list stored in the memory 60 corresponding to the added or removed application is updated. The information processing unit 52 may execute the update of the application list and the operation command list in a case in which the information processing unit 52 executes processing of adding or removing the application.

The first application 62 in the present embodiment is an application (streaming application) for displaying a streaming image. The second application 64 is an application (color Doppler application) for displaying a color Doppler image. In the color Doppler application, the ultrasound diagnostic data such as the B-mode image data and the Doppler data transmitted from the ultrasound diagnostic apparatus 1 to the information processing apparatus 50 over a predetermined number of frames is stored in the memory 60.

The information processing unit 52 that executes the color Doppler application may execute, for example, the following processing described in JP2015-198777A. The information processing unit 52 extracts contour data indicating a contour (pattern) of a heart from the B-mode image data for each frame. The information processing unit 52 obtains a velocity vector of the blood flow at each position in the heart from the Doppler data and the contour data in the predetermined number of frames, and generates, the Doppler image data indicating the velocity vector of the blood flow at each position in the heart. The Doppler image data may be data in which the magnitude of the velocity vector is represented by brightness and a direction of the velocity vector is represented by color. The Doppler image data may be, for example, image data in which the velocity vector in a direction away from the ultrasound probe 10 is represented by a blue pixel and the velocity vector in a direction toward the ultrasound probe 10 is represented by a red pixel. The Doppler image data may be image data in which the velocity vector of each observation position is represented by a figure such as an arrow.

The third application 66 in the present embodiment is an application (AI analysis application) for performing AI analysis. In the AI analysis application, the APP analysis data (another APP analysis data), the ultrasound examination report, and the like obtained by another application are stored in the memory 60. The data stored in the memory 60 may be transmitted from the ultrasound diagnostic apparatus 1 to the information processing apparatus 50. Data indicating a machine learning model necessary for AI analysis (machine learning model data) is stored in the memory 60 in advance by machine learning. In the AI analysis application, whether or not a lesion is generated in the subject is determined based on the other APP analysis data, the ultrasound examination report, the machine learning model data, and the like.

In a case in which the APP analysis data generated by executing the application is image data, the information processing unit 52 outputs the APP analysis data to the analysis image output unit 56. The analysis image output unit 56 transmits the APP analysis data to the image switching unit 18 via the wireless communication by the external wireless unit 72 and the diagnostic apparatus wireless unit 70. The image switching unit 18 outputs the APP analysis data to the display device 40, and the display device 40 displays an image based on the APP analysis data.

The information processing unit 52 may output the APP analysis data sequentially generated with the elapse of time to the analysis image output unit 56 as real-time image data. In this case, the analysis image output unit 56 sequentially transmits the APP analysis data to the image switching unit 18 with the elapse of time, and the image switching unit 18 sequentially outputs the APP analysis data to the display device 40 with the elapse of time. The display device 40 sequentially displays the image based on the APP analysis data output from the image switching unit 18 with the elapse of time, as the real-time image.

The information processing unit 52 may transmit the APP analysis data generated by executing the application to a picture archiving and communication system (PACS) that is a database via the external interface 54 and the external wireless unit 72. The PACS stores the APP analysis data. The controller 20 reads the APP analysis data from the PACS via the diagnostic apparatus interface 28. The controller 20 may execute processing of displaying the information indicated by the APP analysis data on the display device 40.

In addition, the information processing unit 52 may transmit the APP analysis data to the controller 20 via the external interface 54, the external wireless unit 72, the diagnostic apparatus wireless unit 70, and the diagnostic apparatus interface 28. The controller 20 may store the APP analysis data in a memory provided in the controller 20 or a memory connected to the outside of the ultrasound diagnostic apparatus 1. In addition, the display device 40 may display a numerical value and the like indicated by the APP analysis data.

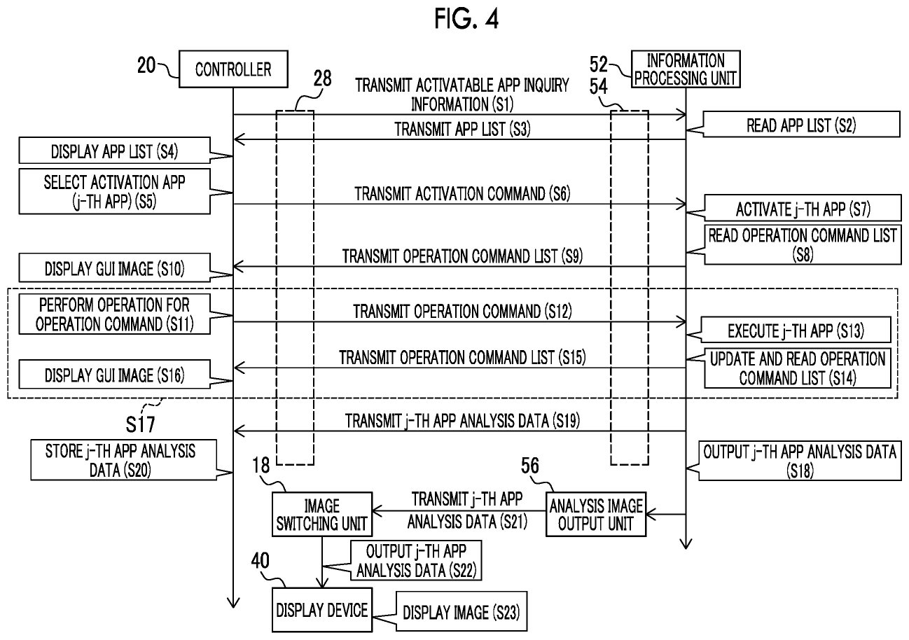

FIG. 4 shows a sequence chart in which the information processing apparatus 50 executes a j-th application as the execution application. j is an integer of any one of 1 to 3. Unless otherwise specified, the communication between the controller 20 and the information processing unit 52 is performed via the diagnostic apparatus interface 28, the diagnostic apparatus wireless unit 70, the external wireless unit 72, and the external interface 54. The notation of “APP” in FIG. 4 is an abbreviation for application.

The controller 20 transmits activatable APP inquiry information for inquiring an activatable application among the applications installed in the information processing apparatus 50, to the information processing unit 52 (S1). In a case in which the activatable APP inquiry information is received, the information processing unit 52 reads the application list from the memory 60 (S2), and transmits the application list to the controller 20 (S3).

In a case in which the application list is received, the controller 20 displays the application list on the display device 40 (S4). In a case in which an operation of selecting the j-th application as the activation application is performed by the user on the operation device 24 while the application list is displayed on the display device 40 or after the display ends (S5), the controller 20 transmits the activation command corresponding to the j-th application to the information processing unit 52 (S6).

In a case in which the activation command is received, the information processing unit 52 activates the j-th application (S7). The information processing unit 52 reads the operation command list for the j-th application from the memory 60 (S8), and transmits the operation command list to the controller 20 (S9).

In a case in which the operation command list is received, the controller 20 constructs the MMI based on the MMI construction information included in the operation command list, and displays the GUI image on the display device 40 (S10). In a case in which the operation for the operation command is performed by the user (S11), the controller 20 transmits the operation command to the information processing unit 52 (S12). In a case in which the operation command is received, the information processing unit 52 executes the j-th application according to the operation command (S13).

The information processing unit 52 updates the operation command list stored in the memory 60 with the execution of the j-th application (S14). The information processing unit 52 reads the updated operation command list from the memory 60 (S14), and transmits the updated operation command list to the controller 20 (S15). In a case in which the operation command list is received, the controller 20 updates the MMI based on the MMI construction information included in the operation command list, and displays the GUI image on the display device 40 (S16).

The operation processing S17 from step S11 to step S16 may be repeated a plurality of times as necessary. In a case in which the operation processing S17 is executed only once, steps S14 to S16 do not have to be executed.

The information processing unit 52 generates j-th APP analysis data by executing the j-th application, and transmits the j-th APP analysis data to the controller 20 (S19). The information processing unit 52 receives and stores the j-th APP analysis data (S20).

In a case in which the j-th APP analysis data is image data, the information processing unit 52 outputs the j-th APP analysis data to the analysis image output unit 56 (S18). The analysis image output unit 56 transmits the j-th APP analysis data to the image switching unit 18 via the external wireless unit 72 and the diagnostic apparatus wireless unit 70 (S21). The image switching unit 18 outputs the j-th APP analysis data to the display device 40 (S22). The display device 40 displays an image based on the j-th APP analysis data (S23).

In a case in which j=1, that is, in a case in which the application is the first application 62, the information processing unit 52 executes the first application 62. The information processing unit 52 sequentially outputs the B-mode image data (first APP analysis data) to the analysis image output unit 56 with the elapse of time, as the data indicating the real-time image (S18). The analysis image output unit 56 sequentially transmits the B-mode image data to the image switching unit 18 with the elapse of time (S21). The image switching unit 18 outputs the B-mode image data to the display device 40 (S22). The display device 40 displays the image based on the B-mode image data (S23). As a result, the B-mode image as the real-time image is displayed on the display device 40.

In a case in which j=2, that is, in a case in which the application is the second application 64, the information processing unit 52 executes the second application 64. The information processing unit 52 generates the color Doppler image data over a plurality of frames as second APP analysis data over a plurality of frames, and stores the color Doppler image data in the memory 60. The information processing unit 52 reads out the color Doppler image data (second APP analysis data) from the memory 60 in time series, and outputs the color Doppler image data to the analysis image output unit 56 (S18). The analysis image output unit 56 transmits the color Doppler image data to the image switching unit 18 in time series (S21). The image switching unit 18 outputs the color Doppler image data to the display device 40 (S22). The display device 40 displays the image based on the color Doppler image data (S23). As a result, the color Doppler images are sequentially displayed on the display device 40 in time series.

In a case in which j=3, that is, in a case in which the application is the third application 66, the information processing unit 52 executes the third application 66 to determine whether or not the lesion is generated in the subject. The information processing unit 52 outputs third APP analysis data indicating a determination result to the analysis image output unit 56 (S18). The analysis image output unit 56 transmits the third APP analysis data to the image switching unit 18 (S21). The image switching unit 18 outputs the third APP analysis data to the display device 40 (S22). The display device 40 displays an image based on the third APP analysis data (S23).

In the ultrasound diagnostic system 100 according to the present embodiment, as the interfaces common to a plurality of applications, the diagnostic apparatus interface 28 and the external interface 54 are constructed in the ultrasound diagnostic apparatus 1 and the information processing apparatus 50, respectively. An interface for transmitting and receiving the information to and from the ultrasound diagnostic apparatus 1 is not individually provided for each of the plurality of applications. The plurality of applications transmit and receive the information to and from the ultrasound diagnostic apparatus 1 via the external interface 54 and the diagnostic apparatus interface 28 that are constructed in common to the plurality of applications.

Therefore, by configuring an application suitable for the external interface 54 and the diagnostic apparatus interface 28, the application can be additionally installed in the information processing apparatus 50. As a result, it is easy to additionally install the application in the information processing apparatus 50.

In addition, the ultrasound diagnostic apparatus 1 according to the embodiment of the present disclosure transmits the ultrasound diagnostic data generated by the transmission and the reception of the ultrasound, to the information processing apparatus 50, and comprises the controller 20 that acquires the external processing data in which the ultrasound diagnostic data is processed, from the information processing apparatus 50. The controller 20 acquires the operation command list as the operation command information for the application for the ultrasound diagnostic data, that is, the application installed in the information processing apparatus 50, from the information processing apparatus 50 (S9). The controller 20 constructs the MMI corresponding to the operation command list.

Further, the information processing apparatus 50 (ultrasound information processing apparatus) according to the embodiment of the present disclosure comprises the information processing unit 52 that acquires the ultrasound diagnostic data generated by the ultrasound diagnostic apparatus 1 by the transmission and the reception of the ultrasound. The information processing unit 52 acquires the activation command from the ultrasound diagnostic apparatus 1 (S6), and transmits the operation command list for the execution application, which is activated by the activation command, among the plurality of applications for the ultrasound diagnostic data to the ultrasound diagnostic apparatus 1 (S9). The operation command list includes the operation command for operating the execution application and the information for constructing the MMI for operating the execution application. The ultrasound diagnostic apparatus 1 constructs the MMI corresponding to the operation command information.

In this way, in the ultrasound diagnostic system 100 according to the present embodiment, the operation command list for the execution application is transmitted from the information processing apparatus 50 to the ultrasound diagnostic apparatus 1. The ultrasound diagnostic apparatus 1 constructs the MMI by itself based on the operation command list. Therefore, even in a case in which the application is newly installed in the information processing apparatus 50, in a case in which the newly installed application is executed as the execution application, the MMI construction information is transmitted from the information processing apparatus 50 to the ultrasound diagnostic apparatus 1. As a result, it is not necessary to newly add the information for constructing the MMI to the ultrasound diagnostic apparatus 1. Therefore, it is easy to additionally install the application in the information processing apparatus 50.

It should be noted that a fourth application for executing the following processing may be additionally installed in the information processing apparatus 50. The information processing unit 52 that executes the fourth application receives the B-mode image data sequentially transmitted from the controller 20 with the elapse of time via the diagnostic apparatus interface 28 and the external interface 54. The B-mode image data sequentially received with the elapse of time indicates a real-time ultrasound image (hereinafter, may be referred to as a real-time ultrasound image). The information processing unit 52 executes processing of extracting the contour of the tissue from the real-time ultrasound image, and drawing the contour of the tissue on the real-time ultrasound image.

The information processing unit 52 generates the contour data indicating the contour of the tissue from the B-mode image data sequentially transmitted with the elapse of time, synthesizes the contour data with the B-mode image data, and sequentially generates contour/B-mode image data with the elapse of time, as fourth APP image data. The contour/B-mode image data is data indicating an image in which a figure indicating the contour (figure in which the contour is drawn by a line) is superimposed on the B-mode image.

In addition, a fifth application for executing the following processing may be additionally installed in the information processing apparatus 50. The image processing executed in the fifth application may be processing (fusion display processing) of showing a basic image such as a magnetic resonance imaging (MRI) image or a computed tomography (CT) image and the real-time ultrasound image side by side for the common observation cross section in the subject. The information processing unit 52 reads the basic image stored in advance in the memory 60. In addition, the information processing unit 52 may read the basic image from an external device. In order to execute the fusion display processing, for example, the ultrasound probe 10 is provided with a position sensor. Based on a position of the ultrasound probe 10 detected by the position sensor, the basic image in the cross section in which the real-time ultrasound image is observed is displayed on the display device 40 side by side with the real-time ultrasound image.

The information processing unit 52 synthesizes an image indicating the basic image data and the B-mode image data, and sequentially generates basic/B-mode image data in which the basic image and the B-mode image are shown side by side with the elapse of time, as fifth APP image data.

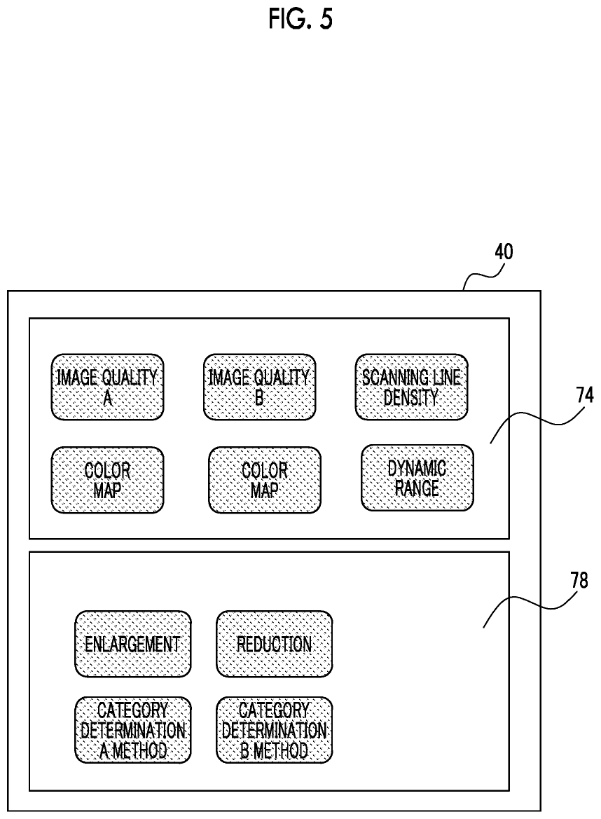

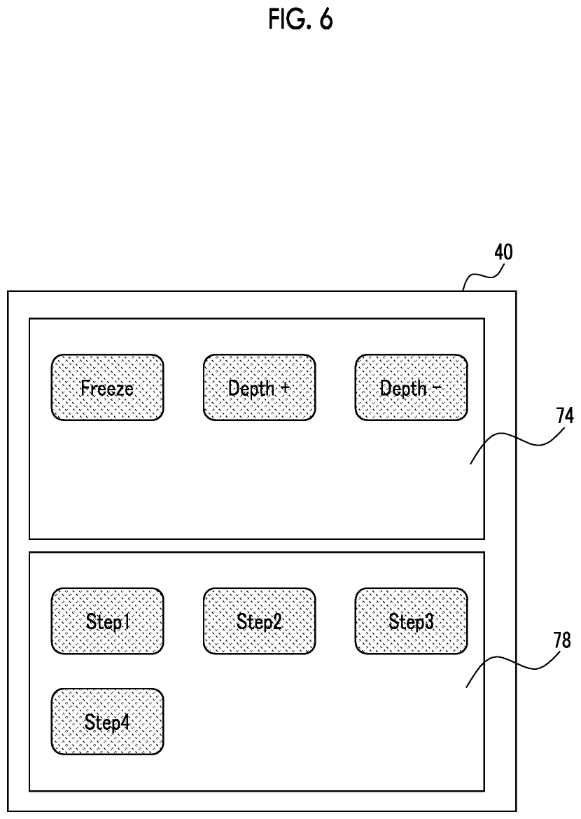

FIGS. 5 and 6 show an example of the GUI image displayed on the display device 40 of the ultrasound diagnostic apparatus 1 in steps S10 and S16 of FIG. 4. In FIGS. 5 and 6, the applications executed by the information processing apparatus 50 are different from each other. As shown in FIGS. 5 and 6, in a diagnostic apparatus command area 74 on an upper part of a screen of the display device 40, an ultrasound diagnostic command is displayed as a command of a function provided in the ultrasound diagnostic apparatus 1. In an information processing apparatus command area 78 on a lower part, a button is displayed as the operation portion of the operation command in a case in which the application is executed. In a case in which the display device 40 is a touch panel, the button of each command is operated by the user touching the button with a finger. In a case in which the display device 40 is a display, the button of each command is operated by a mouse or the like being clicked after a cursor is set thereon by the mouse or the like.

In the diagnostic apparatus command area 74 in FIG. 5, as buttons for adjusting an image quality of the image displayed on the display device 40, a button noted as “image quality A” and a button noted as “image quality B” are shown. In addition, as buttons for adjusting a scanning line density or a dynamic range, a button noted as “scanning line density” and a button noted as “dynamic range” are shown. In addition, as a button for displaying a color map based on Doppler measurement, a button noted as “color map” is shown.

In the information processing apparatus command area 78 in FIG. 5, as buttons for enlarging and reducing the image, buttons noted as “enlargement” and “reduction” are shown.

In addition, as buttons for selecting a determination method according to a medical guideline, buttons noted as “category determination A method” and “category determination B method” are shown.

In the diagnostic apparatus command area 74 in FIG. 6, as a button for freezing the real-time image, a button noted as “Freeze” is shown. In addition, as buttons for adjusting a depth of focus of the ultrasound beam, two buttons noted as “Depth+” and “Depth−” are shown. In the information processing apparatus command area 78 in FIG. 6, as buttons for executing each operation step of the application, four buttons noted as “Step 1”, “Step 2”, “Step 3”, and “Step 4” are shown.

In steps S9 and S15 of FIG. 4, the operation command list transmitted from the information processing unit 52 to the controller 20 includes the MMI construction information for the ultrasound diagnostic apparatus 1 to construct the man-machine interface (MMI). The MMI may be configured by a combination of the GUI image displayed on the display device 40 and the operation device 24. In addition, in a case in which the display device 40 is a touch panel, the GUI may be a button, an image, a text, or the like displayed on the display device 40.

In a case in which the operation command list transmitted from the information processing unit 52 in step S6 of FIG. 4 is received, the controller 20 extracts the MMI construction information from the operation command list. The controller 20 displays the GUI image on the display device 40.

The MMI construction information includes an ID for specifying a display target such as a button, an image, or a text. In a case in which the display target is a button, the MMI construction information may include a display title, a length in a vertical direction, a length in a horizontal direction, a position displayed on the display device 40, a shape (rectangular shape, circular shape, triangular shape, or the like), an RGB code indicating a color, information indicating whether or not the button can be operated, information indicating whether or not the button is for executing the ultrasound diagnostic command, information indicating whether or not the operation command is executed, and the like.

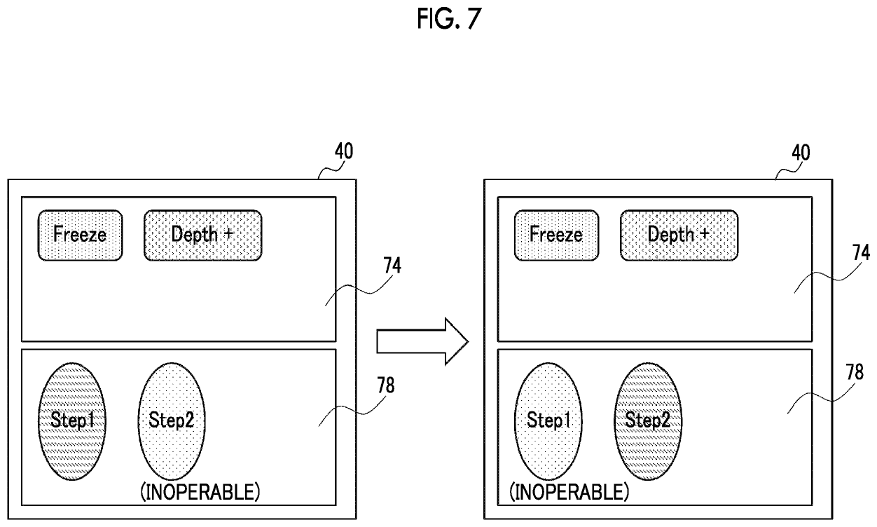

FIG. 7 shows two examples of the GUI image displayed on the display device 40. In the diagnostic apparatus command area 74 of the right and left images, as the button for freezing the real-time image, the button noted as “Freeze” is shown. In addition, as the button for adjusting a depth of focus of the ultrasound beam, the button noted as “Depth+” is shown. In addition, in the information processing apparatus command area 78 of the right and left GUI images, as the buttons for executing each operation step of the application, two buttons noted as “Step 1” and “Step 2” are shown. In the left GUI image, “inoperable” is shown below the button noted as “Step 2”, which indicates that the button noted as “Step 2” cannot be pressed. In the right GUI image, “inoperable” is shown below the button noted as “Step 1”, which indicates that the button noted as “Step 1” cannot be pressed.

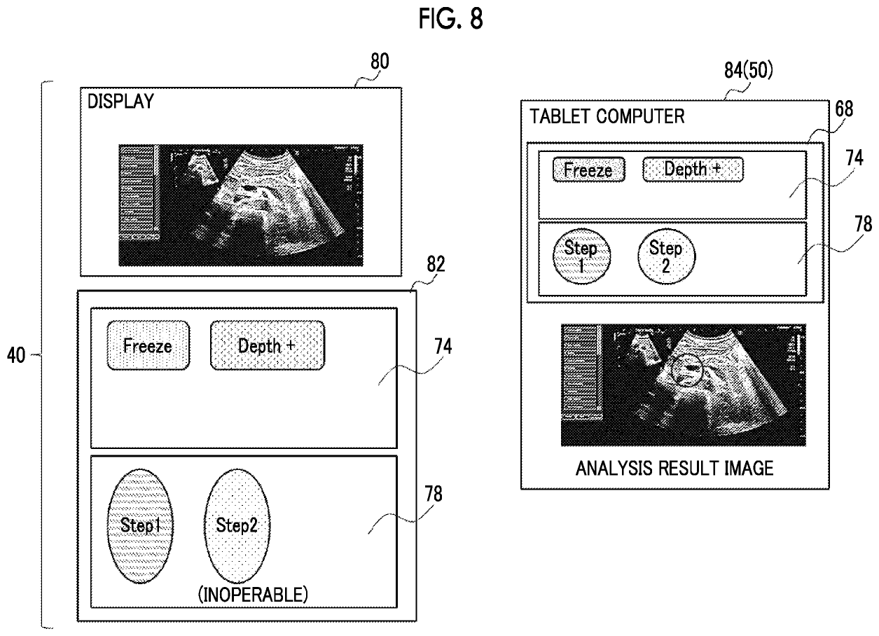

FIG. 8 shows an example in which the display device 40 is configured by a display 80 and a touch panel 82, and the information processing apparatus 50 is a tablet computer 84. FIG. 8 shows an example of the GUI image displayed on each of the display 80, the touch panel 82, and the tablet computer 84. In this example, the information processing unit 52 displays an image including a part or all of the images displayed by the ultrasound diagnostic apparatus 1 on the display equipment 68 of the tablet computer 84. The B-mode image is shown on the display 80. The B-mode image may be a still image or a moving image. On the tablet computer 84, the same buttons (operation portions) as the buttons in the diagnostic apparatus command area 74 and the information processing apparatus command area 78 displayed on the touch panel 82 of the ultrasound diagnostic apparatus 1 are shown. That is, the tablet computer 84 is constructed with the same GUI as the GUI constructed in the ultrasound diagnostic apparatus 1. Further, the tablet computer 84 is shown an analysis result image showing an analysis result of the application. In the example shown in FIG. 8, the B-mode image in which an area in which the lesion is recognized is marked with a circle is shown.

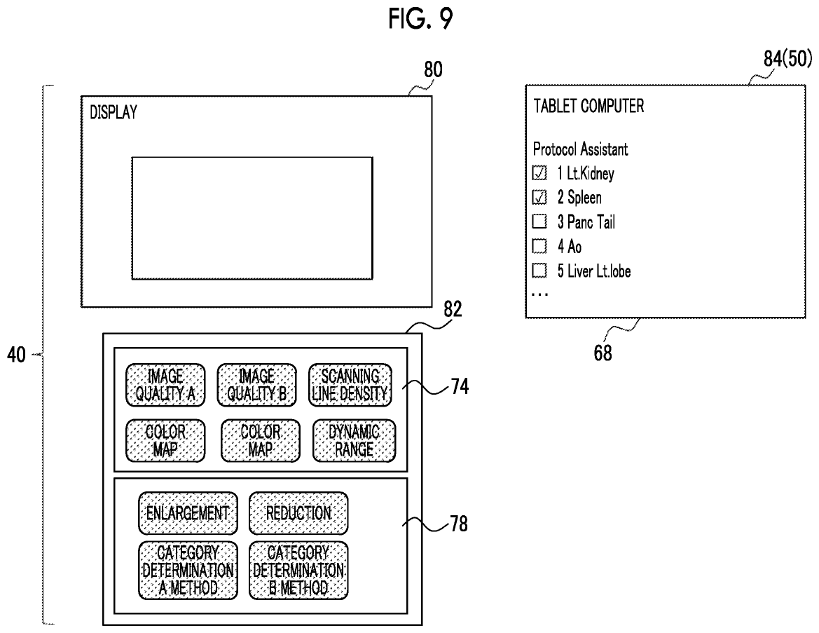

FIG. 9 shows an example of the GUI image displayed on each of the display 80, the touch panel 82, and the tablet computer 84 in a case in which the display device 40 is configured by the display 80 and the touch panel 82, and the information processing apparatus 50 is the tablet computer 84. The B-mode image is shown on the display 80. The display 80 may display an image that is normally displayed in a case in which the subject is diagnosed by the ultrasound diagnostic apparatus 1, for example, the ultrasound image such as the B-mode image or the color Doppler image. An image showing the operation of the ultrasound diagnostic apparatus 1 may be displayed. An operation procedure (manual) for each operation command displayed on the touch panel 82 of the ultrasound diagnostic apparatus 1 is displayed on the tablet computer 84.

The configurations of the present disclosure are described below.

### Configuration 1:

An ultrasound information processing apparatus comprising: an information processing unit that acquires ultrasound diagnostic data generated by an ultrasound diagnostic apparatus by transmission and reception of ultrasound, in which the information processing unit acquires an activation command from the ultrasound diagnostic apparatus, and transmits operation command information for an execution application, which is activated by the activation command, among a plurality of applications for the ultrasound diagnostic data to the ultrasound diagnostic apparatus, the operation command information includes an operation command for operating the execution application and information for constructing a man-machine interface for operating the execution application, and the ultrasound diagnostic apparatus constructs the man-machine interface corresponding to the operation command information.

### Configuration 2:

The ultrasound information processing apparatus according to configuration 1, in which the man-machine interface provides an operation portion used with execution of the execution application to a user.

### Configuration 3:

The ultrasound information processing apparatus according to configuration 1 or 2, in which the operation command information includes information for constructing a graphical user interface used with execution of the execution application, and the ultrasound diagnostic apparatus constructs the graphical user interface as the man-machine interface.

### Configuration 4:

The ultrasound information processing apparatus according to any one of configurations 1 to 3, in which the information processing unit displays an operation procedure for the operation command on display equipment.

### Configuration 5:

The ultrasound information processing apparatus according to any one of configurations 1 to 4, in which the information processing unit displays an image including a part or all of images displayed by the ultrasound diagnostic apparatus on display equipment.

### Configuration 6:

An ultrasound diagnostic system comprising: the ultrasound information processing apparatus according to any one of configurations 1 to 5; and the ultrasound diagnostic apparatus.

### Configuration 7:

An ultrasound diagnostic apparatus comprising: a controller that transmits ultrasound diagnostic data generated by transmission and reception of ultrasound to an information processing apparatus, and acquires external processing data in which the ultrasound diagnostic data is processed from the information processing apparatus, in which the controller acquires operation command information for an application for the ultrasound diagnostic data, which is installed in the information processing apparatus, from the information processing apparatus, and constructs a man-machine interface corresponding to the operation command information.

### Configuration 8:

The ultrasound diagnostic apparatus according to configuration 7, in which the man-machine interface provides an operation portion used with execution of the application to a user.

### Configuration 9:

The ultrasound diagnostic apparatus according to configuration 7 or 8, in which the operation command information includes information for constructing a graphical user interface used with execution of the application, and the controller constructs the graphical user interface as the man-machine interface.

### Configuration 10:

The ultrasound diagnostic apparatus according to any one of configurations 7 to 9, in which the controller displays an image including a part or all of images displayed by the ultrasound diagnostic apparatus on the information processing apparatus.

What is claimed is:

1. An ultrasound information processing apparatus comprising:
an information processing unit that acquires ultrasound diagnostic data generated by an ultrasound diagnostic apparatus by transmission and reception of ultrasound,
wherein the information processing unit
acquires an activation command from the ultrasound diagnostic apparatus, and
transmits operation command information for an execution application, which is activated by the activation command, among a plurality of applications for the ultrasound diagnostic data to the ultrasound diagnostic apparatus,
the operation command information includes an operation command for operating the execution application and information for constructing a man-machine interface for operating the execution application, and
the ultrasound diagnostic apparatus constructs the man-machine interface corresponding to the operation command information.

2. The ultrasound information processing apparatus according to claim 1,
wherein the man-machine interface provides an operation portion used with execution of the execution application to a user.

3. The ultrasound information processing apparatus according to claim 1,
wherein the operation command information includes information for constructing a graphical user interface used with execution of the execution application, and
the ultrasound diagnostic apparatus constructs the graphical user interface as the man-machine interface.

4. The ultrasound information processing apparatus according to claim 1,
wherein the information processing unit displays an operation procedure for the operation command on display equipment.

5. The ultrasound information processing apparatus according to claim 1,
wherein the information processing unit displays an image including a part or all of images displayed by the ultrasound diagnostic apparatus on display equipment.

6. An ultrasound diagnostic system comprising:
the ultrasound information processing apparatus according to claim 1; and
the ultrasound diagnostic apparatus.

7. An ultrasound diagnostic apparatus comprising:
a controller that
transmits ultrasound diagnostic data generated by transmission and reception of ultrasound to an information processing apparatus, and
acquires external processing data in which the ultrasound diagnostic data is processed from the information processing apparatus,
wherein the controller acquires operation command information for an application for the ultrasound diagnostic data, which is installed in the information processing apparatus, from the information processing apparatus, and constructs a man-machine interface corresponding to the operation command information.

8. The ultrasound diagnostic apparatus according to claim 7,
wherein the man-machine interface provides an operation portion used with execution of the application to a user.

9. The ultrasound diagnostic apparatus according to claim 7,
wherein the operation command information includes information for constructing a graphical user interface used with execution of the application, and
the controller constructs the graphical user interface as the man-machine interface.

10. The ultrasound diagnostic apparatus according to any one of claim 7,
wherein the controller displays an image including a part or all of images displayed by the ultrasound diagnostic apparatus on the information processing apparatus.
